(19) United States
(12) Patent Application Publication
(10) Pub. No.: US 2006/0293218 A1
(43) Pub. Date: Dec. 28, 2006

(54) Salicylate therapeutic compound and process for controlled delivery thereof

(76) Inventors: Jay M. Meythaler, Grosse Pointe, MI (US); Jean Peduzzi Nelson, Northville, MI (US); Landon C.G. Miller, Tuscaloosa, AL (US)

Correspondence Address: GIFFORD, KRASS, GROH, SPRINKLE & CITKOWSKI, P.C, PO BOX 7021, TROY, MI, 48007-7021, US

(21) Appl. No.: 11/333,008

(22) Filed: Jan. 17, 2006

Related U.S. Application Data
(63) Continuation of application No. 11/158,679, filed on Jun. 22, 2005, now abandoned.

(51) Int. Cl. A61K 38/04; A61K 31/7024; A61K 31/717; A61K 31/60; A61K 31/192; A61F 13/02

(52) U.S. Cl. 514/2; 514/25; 514/159; 514/57; 424/448; 514/568

(57) ABSTRACT

A therapeutic composition includes a salicylate administering concert with at least one salicylate excretion compound of a glycine salicylate conjugate and a gluconoride salicylate conjugate. A therapeutic composition is provided that has an extended excretion half-life for a salicylate that includes the administration of the salicylate in concert with an enzymatic substrate competitor. The enzymatic substrate competitor being competitive with salicylate or gluconoride. Salicylate enzymatic substrate competitors include benzoate or other aromatic derivatives, while gluconoride enzymatic substrate competitors include an active phenolic unit. A process for treating a human or non-human subject includes administering to a subject a therapeutically effective amount of a salicylate and a salicylate pharmacokinetics modifier of glycine salicylate conjugate, gluconoride salicylate conjugate, benzoate, an aromatic derivative and an active phenolic unit, along with a pharmaceutically acceptable carrier.

### FIELD OF THE INVENTION

The present invention in general relates to non-acetylated salicylate compounds, and in particular to delivery of such compounds under conditions to enhance therapeutic efficacy.

### BACKGROUND OF THE INVENTION

Choline salicylate is a well-known analgesic that met with limited acceptance owing to a foul taste associated with oral delivery and perturbations to endogenous choline levels after discontinuation of the drug. The attractive attributes of salicylates have been slow metabolic clearing and less of a propensity to induce gastric irritation and bleeding. These attributes of salicylates are in marked contrast to acetylated salicylates, of which aspirin is the preeminent member.

Choline salicylate became a more attractive oral analgesic after compounding with salicylate salts such as magnesium salicylate. The resulting mixed choline-salt salicylate can take various forms such as choline magnesium salicylate and choline magnesium trisalicylate. Regardless of stoichiometry of the choline-metal salt salicylate, the resulting compound is a powder amenable to forming into tablets. Formulation as a tablet addresses in part the limitations of choline salicylate, yet retains a comparatively high concentration of choline. While choline is generally considered to have mild side effects, cholinergic receptors associated with peripheral tissues such as stomach fundus, urinary bladder, trachea and nicotinic receptors throughout an individual potential concern as side effects and an interference to simultaneous treatments. I.H. Ulus et al., Biochemical Pharmacology, 37(14):2747-55 (1988). Additionally, high choline dosing in some individuals has been associated with symptoms of clinical depression.

Thus, there exists a need for salicylate-containing therapeutic compositions for indications such as osteoarthritis, rheumatoid arthritis, muscle pain, anti-inflammatories, analgesics, antipyretics and in the treatment of traumatic brain injury with superior control over elimination half-life, mode of delivery, and the amount of choline relative to salicylate in a formulation.

### SUMMARY OF THE INVENTION

A therapeutic composition includes a salicylate administering concert with at least one salicylate excretion compound of a glycine salicylate conjugate and a gluconoride salicylate conjugate. A therapeutic composition is provided that has an extended excretion half-life for a salicylate that includes the administration of the salicylate in concert with an enzymatic substrate competitor, the enzymatic substrate competitor being competitive with salicylate or gluconoride. Salicylate enzymatic substrate competitors include benzoate or other aromatic derivatives, while gluconoride enzymatic substrate competitors include an active phenolic unit.

A process for treating a human or nonhuman subject includes administering to a subject a therapeutically effective amount of a salicylate and a salicylate pharmacokinetics modifier of glycine salicylate conjugate, gluconoride salicylate conjugate, benzoate, an aromatic derivative and an active phenolic unit, along with a pharmaceutically acceptable carrier.

### DETAILED DESCRIPTION OF THE PREFERRED EMBODIMENTS

The present invention has utility as a therapeutic composition indicated for chronic and acute pain associated with ailments illustratively including osteoarthritis, rheumatoid arthritis, as a general analgesic, and as antipyretic. The inventive compositions have the attribute of not affecting platelet aggregation and as such are also indicated in instances of cerebrospinal swelling associated with traumatic brain injury.

An inventive salicylate composition includes a mixture of salicylates that allows one to adjust the time of peak dosage after administration, the half-time elimination or a combination thereof. In another embodiment, the present invention affords a single salicylate compound, or a mixture of salicylates in a hydrophilic polymer matrix, that shifts the peak dosage after administration to a latter time relative to the comparable dose absent the matrix. The present invention yields a tunable dosing profile.

As used herein, “bioequivalence” is defined to mean the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. See Code of Federal Regulations, Title 21, Apr. 1, 1997 edition, Part 320.1, Definitions (e) Bioequivalence, page 195.

While the present invention is principally disclosed herein with respect to solid oral delivery dose, it is appreciated that inventive salicylate compositions are readily prepared in forms other than a solid oral delivery dose to treat a subject. As used herein, a “subject” is defined to include mammals and birds inclusive of humans. Examples of nonhuman subjects illustratively include cows, dogs, cats, sheep, goats, pigs, and chickens. One skilled in the art will readily appreciate that an inventive mixture of salicylates, alone or in combination with enzymatic substrate competitors, are readily formulated as a topical rubefacient, transdermal patch, intravenous solution, intramuscular solution, intrathecal solution, intraventricular solution, a suppository, an oral solution, and a nasal spray. Such delivery forms typically include a physiologically acceptable sterile aqueous or non-aqueous solution, dispersion, suspension or emulsion or a sterile powder for reconstitution into a sterile injectable solution or dispersion. Examples of suitable aqueous and non-aqueous carriers, diluents, solvents and vehicles illustratively include water; ethanol; polyols such as propylene glycol, polyethylene glycol, and glycerol; suitable mixtures thereof; vegetable oils such as olive oil; and injectable organic esters such as ethyloleate. Adjuvants are typically provided to facilitate delivery and storage. Typical adjuvants illustratively include osmolarity buffers such as saline; pH buffers; antioxidants; and antimicrobials: the identity and typical quantities of such adjuvants are detailed in Remington's Pharmaceutical Sciences, 20^{th} Edition, pp. 240-241, 1015-1017, 1510-1530, and 1551-1555.

An inventive salicylate composition exhibits an approximate in vivo hysteresis profile when the plasma curves at less than saturation dosing for salicylate is deconvoluted. J.G. Wagner et al., J. Pharm. Sciences, 52:610-611 (1963). The absorption profile for an inventive formulation is approximately characterized by three general phases; I, II, and III. Phase I is characterized by the initial time period where minimal absorption of salicylate occurs. In conventional salicylate dosing such as with choline magnesium salicylate tablets, phase I is on the range of 15 to 30 minutes. Phase II is an exponential period, following phase I during which most of the absorption of salicylate occurs. In conventional salicylate dosing such as with choline magnesium salicylate tablets, phase II yield as peak dose after 1 to 2 hours. Phase III involves the time period when salicylate absorption ends and is treated theoretically as asymptotically diminishing to a complete absorption. In conventional salicylate dosing such as with choline magnesium salicylate tablets, complete absorption is obtained in 10 to 20 hours.

Through the use of a hydrophilic polymer matrix as part of the present invention, phase I is extended to occur from 1 to 5 hours with a mean of about 2.7 hours after ingestion. Phase II is adjusted to occur from S to 12 hours with a mean of about 8 hours after phase II. Phase III is adjusted to occur from 12 to about 16 hours with a mean of about 14 hours after ingestion. The net result is that salicylate in conventional form that typically is predominantly absorbed from the stomach, is absorbed according to the present invention predominantly in the duodenum. The actual time being modified by factors illustratively including subject mass, stomach contents, metabolic rate, age, and dose. A typical daily dose of salicylate according the present invention for an adult is between 300 and 900 milligrams (mg) per day exclusive of the weight of counter ions, and for children a typical dose ranges from 12 to 50 mg/kilogram (kg)/day exclusive of the weight of counter ions. In terms of weight percentages, up to 25% and preferably about 8% of the total dose is absorbed during phase I. During phase II between about 75% and 100% and preferably about 90% is absorbed during phase II, with the remainder, if any, being absorbed during phase III. It is appreciated that salicylate excretion is via a renal glycine conjugate (salicyluric acid) and a renal phenolic glucuronide conjugate. Excretion is rate limited by the hepatic formation of the excretion conjugates. Glucuronide conjugate in bile is capable of reabsorption, thereby slowing the net rate of excretion. As a result of the known excretion pathways and kinetics, one skilled in the art will appreciate that there is a dose dependency to the excretion metabolite ratio of glycine: glucuronide conjugates, as well as the amount of glucuronide reabsorption.

In a first inventive embodiment, one or more soluble salicylate therapeutic species is combined in an oral drug delivery form with a hydrophilic polymer matrix swelling agent that extends the duration of drug release profile phases compared to the bare one or more soluble salicylate therapeutic species. Oral drug delivery forms operative herein illustratively include tablets, capsules, and dragees. It is appreciated that the specific salicylate and the amount of swelling agent are preselected in order to control the time profile release of the salicylate. It is appreciated that the amount of the hydrophilic polymer matrix swelling agent varies depending upon factors such as the nature of the salicylate, grain size of the salicylate, the physical properties of the swelling agent, and the desired the time release profile, it is preferred to employ amounts of the salicylate to provide the desired dosing effect. Optionally, a hydrophilic polymer matrix swelling agent is replaced with or used in combination with a gel, permeable membrane, osmotic system such as those of Alza Corp. (Mountain View, Calif.), microparticles, liposomes, or microspheres. As is known in the art, such hydrophilic polymer matrix swelling agents and amounts thereof, are preselected in order to control the time release of salicylate.

Hydrophilic polymer matrix swelling agents operative herein illustratively include polymers such as carboxy C₁-C₈ alkylcelluloses; C₁-C₈ alkylcelluloses; and waxes such as beeswax; and natural materials such as gums or gelatins or mixtures of any of the above. A preferred swelling agent is hydroxypropyl methylcellulose, in an amount ranging from about 5% to about 50% parts by weight per 100 parts by weight of salicylate, where the salicylate weight is determined independent of counter ion or conjugate moiety. The preferred quantity is chosen to afford sustained time release over a period corresponding to phase II of 8 to 12 hours as demonstrated by in vitro dissolution techniques known to the art.

A binder is optional included in an inventive composition. While any known binding material is operative herein, a preferred binder is a polymer incorporating the repeating unit of 1-ethenyl-2-pyrrolidinone. These polymers generally have molecular weights of from 10,000 and 700,000, and are also known as commercially as povidones. Typically, povidones are present from 1 to 5 % parts by weight per 100 parts of salicylate, where the salicylate weight is determined independent of counter ion or conjugate moiety.

Conventional processing aids such as lubricants and desiccants are appreciated to be operative herein. Typical lubricants illustratively include stearic acid and salts thereof and are used in a manner as known to the art. Remington's Pharmaceutical Science, 20^{th} Edition. Desiccants are also conventional to oral solid dose compounding and illustratively include silicon dioxide and starches. Lubricants and desiccants are typically present from 0.05 to 2 % parts by weight per 100 parts of salicylate, where the salicylate weight is determined independent of counter ion or conjugate moiety.

Salicylates operative in the present invention include a variety of physiologically compatible counter ions and conjugates and illustratively include alkali metal ion salicylates such as lithium, sodium, and potassium; alkali earth ion salicylates such as magnesium, and calcium; transition metal salicylates such as iron; other metal ions such as aluminum; quaternary alkyl ammoniums such as tetra (C₂-C₈) ammoniums; organics such as choline; amino acids such as glycine (synonymously known as salicyluric acid), alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, omithine and lysine, hydroxylysine, citrulline, kynurenine, (4-aminophenyl)alanine, 3-(2′-naphthyl)alanine, 3-(1+-naphthyl)alanine, methionine sulfone, (t-butyl)alanine, (t-butyl)glycine, 4-hydroxyphenyl-glycine, aminoalanine, phenylglycine, vinylalanine, propargyl-gylcine, 1,2,4-triazolo-3-ala, 3-aminotyrosine, trifluoromethylalanine, 2-thienylalanine, (2-(4-pyridyl)ethyl)cysteine, 3,4-dimethoxy-phenylalanine, 3-(2′-thiazolyl)alanine, ibotenic acid, 1-amino-1-cyclopentane-carboxylic acid, 1-amino-1-cyclohexanecarboxylic acid, quisqualic acid, 3-(trifluoromethylphenyl)alanine, (cyclohexyl)glycine, thiohistidine, 3-methoxytyrosine, norleucine, norvaline, alloisoleucine, homoarginine, thioproline, dehydroproline, hydroxyproline, homoproline, indoline-2-carboxylic acid, 1,2,3,4-tetrahydroisoquinoline-3-carboxylic acid, 1,2,3,4-tetrahydroquinoline-2-carboxylic acid, a-amino-n-butyric acid, cyclohexylalanine, 2-amino-3-phenylbutyric acid; oligopeptides composed of from 2 to 5 of the above amino acids; heterocyclics such as glucuronide, glucuronamide, aminopyridines, and endocannibinoids, endorphins, enkephalins and dynorphins; multiple species adducts such as choline magnesium salicylate, and choline magnesium trisalicylate; and combinations thereof. Particularly preferred salicylates according to present invention include choline magnesium salicylate, choline magnesium trisalicylate, glycine salicylate and glucuronide salicylate.

In an alternate embodiment, the half-life of excretion is adjusted independent of the release rate as modified by the addition of a swelling agent. Excretion half-life is modified according to the present invention through a recognition that hepatic capacity to modify a salicylate into one of the two predominant excreted forms of a glycine salicylate or glucuronide salicylate is limited. Further, it is appreciated that the glucuronide salicylate excretion pathway is slowed by reabsorption of glucuronide salicylate from bile.

According to the present invention, excretion rates are increased by the administration of glycine with a therapeutic quantity of salicylate. Without intending to be bound to a particular theory, the addition of glycine appears to assure that serum glycine level is not the rate-limiting step in formation of the salicyluric acid. In a particular embodiment having a reduced half-life of excretion, the salicylate is administered in concert with free glycine or a glycine-rich protein such as gelatin. Typically, a molar stoichiometric ration of glycine:salicylate of between 0.2-2:1 is used in the present invention with greater relative quantities of glycine being used in instances where high end doses are administered and/or rapid excretion is desired. According to the present invention, a single 570 mg dose of salicylate with a 2:1 molar ratio of glycine:salicylate is cleared in 40 to 70% of the time required for the same salicylate dose given independent of glycine, with the variability in excretion half-life being related to those factors such as subject mass, stomach contents, metabolic rate, and age. It is appreciated that the presence of glucuronide in a form illustratively including a glucuronate acid or salt, such as sodium glucuronate within an inventive composition further facilitates excretion via the secondary glucuronide pathway, notwithstanding the partial reabsorption noted in bile. Maximal rates of excretion are noted in the present invention when glucuronide is present at from 10 to 20 molar percent of the quantity of glycine present in an inventive therapeutic dose. It is appreciated that a rapid excretion form of the present invention is well suited for a subject having an indication justifying salicylate therapy yet is under a complex regimen of pharmaceutical treatments with concerns about possible drug interactions. By way of example, an inventive salicylate composition taken in the evening that affords pain relief to allow a restful night and clearance by morning to facilitate the ingestion of a secondary therapeutic is a value in the treatment of a subject suffering multiple chronic ailments. It is appreciated that a rapid excretion clearance composition according to the present invention is readily coupled with a swelling agent as detailed above to induce a delayed release, thereby affording a delayed release and rapid clearance formulation.

In a long-acting embodiment of the present invention, a competitive substrate is provided for hepatic enzymes that form salicylate excretion products. A preferred competitive substrate for both glycine- and glucuronide-renal clearance conjugates is a benzoate ion derivative. Benzoate derivatives operative herein include benzoate salts, benzalkoniums, phthalates, benzaldehydes, and substituted forms thereof where a hydryl moiety attached to the phenyl ring is substituted with a substitute including C₀-C₄ hydroxyl, C₀-C₄ sulfonyl, C₀-C₄ carboxyl, C₁-C₄ trialkyl C₀-C₄ amino and C₂-C₈ quaternary C₀-C₄ amino. Without intending to be bound by a particular theory, it is believed that benzoate aromatic derivatives compete effectively with salicylate as a substrate for enzymatic modification thereby slowing the rate of modification of salicylate to an excretable glycine or glucuronide conjugate. Benzoate counter ions operative herein illustratively include alkali metal ion salts such as lithium, sodium, and potassium; and alkali earth ion salts such as magnesium, and calcium and other biocompatible counter ions and conjugates.

Typically, a molar stoichiometric ratio of benzoate derivative:salicylate of between 0.3-10:1 is used in the present invention with greater relative quantities of benzoate being used in instances where slower excretion is desired. Preferably, the molar stoichiometric ratio of benzoate derivative:salicylate is between 0.5-4:1. According to the present invention, a single 570 mg dose of salicylate with a 3:1 ratio of benzoate derivative:salicylate is cleared in 140 to 340% of the time required for the same salicylate dose given independent of benzoate derivative, with the variability in excretion half-life being related to those factors such as subject mass, stomach contents, metabolic rate, and age.

It is appreciated that an independent and less pronounced competitive substrate according to the present invention as compared to benzoate derivative is a phenolic compound containing active phenolic unit. As used herein “an active phenolic unit” is defined to include a molecule or a subunit of polymeric molecule containing a six-membered aromatic ring moiety having at least one hydroxyl substituent thereon, and having an in vivo solubility sufficient to allow interaction with hepatic enzymes that bind phenol.

Without intending to be bound by a particular theory, it is believed that active phenolic units compete effectively with glucuronide as a reagent for enzymatic coupling to salicylate thereby slowing the rate of modification of salicylate to an excretable glucuronide conjugate. Phenolic compounds operative herein illustratively include phenolates; flavenols, flavanones, catechins; anthrocyanins, such as fruit and vegetable colorings; isoflavones, such as genistein, diadzein; dihydroflavonols; chalcones; quercetins; phenolic acids, such as ellagic acid, tannic acid, and vanillin; hydroxycinnamic acid derivatives, such as caffeic acid, chlorogenic acid, ferulic acid, curcumins, and courmarins; and lignans. It is appreciated that phenolic compounds according to the present invention are routinely found as phytochemicals. As such, it is appreciated that diet and nutritional supplements are implicated in the importance of the glucuronide renal excretion pathway. Exemplary phenol compounds used herein include tyrosine and virgin pressed olive oil containing the non-polar phenols oleuropein- and ligstroside- aglycones and the polar phenols, hydroxytyrosol and tyrosoll. Typically, a phenolic competitive substrate is provided at a molar stoichiometric ratio of active phenolic unit: salicylate of between 0-3:1 with greater relative quantities of phenolics being present in instances where slower excretion is desired. Preferably, the molar stoichiometric ratio of active phenolic unit:salicylate is between 0.05-0.8:1. The variations in ratio of active phenolic unit to salicylate are a result in part of: the competitive bind rate of the active phenolic unit relative to glucuronide as measured by the Michaelis-Menten equation, and the salicylate dosing since excretion kinetics vary in the exponent dependency on salicylate concentration as a function of the salicylate concentration.

It is appreciated that benzoate derivatives and phenolic competitive substrates are optionally combined within a therapeutic dose with the understanding that benzoate tends to slow excretion by both glycine and glucuronide conjugate renal pathways, while an active phenolic unit functions preferentially to slow excretion by the glucuronide pathway. Maximal rates of excretion are noted in the present invention when glucuronide is present at from 10 to 20 molar percent of the quantity of benzoate derivatives present in an inventive therapeutic dose.

An additional aspect involves formulating granules of inventive compositions that vary between granules in at least one dose profile parameter of absorption onset, peak dose, or steady state excretion half-life. It is appreciated that combining such granules into a delivery device affords tunable control over the dose profile parameters of the aggregated dose within the delivery device.

An inventive salicylate composition, alone or in combination with enzymatic substrate competitors for delivery by a variety of routes, including topical, intravenous, intramuscular, intraspinal, intraventricular, anal or intranasal through the use of conventional pharmaceutical formulary. By way of example, injectable forms of an inventive composition range from soluble to highly soluble in aqueous solution and as such, are readily dissolved in sterile saline or other physiological solutions for injectable routes including intravenous, intramuscular, intrathecal and intraventricular.

An inventive salicylate composition, alone or in combination with enzymatic substrate competitors, is readily formulated as a suppository through compounding with a suitable base as detailed in Remington's Pharmaceutical Sciences, 20^{th} Edition, pp. 852-856.

An inventive salicylate composition, alone or in combination with enzymatic substrate competitors, is also recognized to be readily formulated into a topical delivery composition. Suitable bases for inclusion of the inventive composition active ingredients include essentially any base topical formulation. Representative base formulations for an inventive topical composition are found in U.S. Pat. Nos. 3,880,996; 4,775,667; 5,223,257; and 5,223,267. Additional topical base formulations are also disclosed in WIPO Publication WO 02/098404 A1.

An inventive mixture of salicylates alone or in combination with enzymatic substrate competitors or a single salicylate in combination with an enzymatic substrate competitor is also readily formulated into a transdermal patch. A representative transdermal delivery system suitable for delivery of an inventive composition is detailed in U.S. Pat. No. 5,658,587, column 15, line 23 -column 22, line 53.

### EXAMPLES

In order to demonstrate the effectiveness of the compositions and method of the present invention over known conventional salicylate compositions, a number of compositions are prepared according to the invention as oral tablets and compared to action of a conventional formulation in a model human adult subject. The bioequivalence of soluble salicylates used herein is assumed for the purposes of the subsequent calculations. Dosing data for a single dose of each composition is also provided, where times are provided in hours. Excretion half-life is calculated based on the steady state administration of 4 doses administrated once daily for 4 consecutive days. The compositions are summarized in Table 1 where quantities are provided in milligrams.

TABLE 1
Test Tablet Composition

| | FORMULATION | | | | | | | |
|---|---|---|---|---|---|---|---|---|
| INGREDIENT | A | B | C | D | E | F | G | Comparative |
| Salicylate | 570 | 570 | 570 | 570 | 570 | 570 | 570 | 570 |
| Salicylate conjugate | Choline Mg | CM | CM | CM | CM | Glycine | CM | CM |
| (identity and weight) | (CM) | 180 | 180 | 180 | 180 | 308 | 180 | 180 |
| | 180 | | | | | | | |
| Hydroxypropyl | 205 | — | — | — | — | 205 | 205 | |
| methylcellulose | | | | | | | | |
| Sodium benzoate | — | 1500 | 1500 | | | | 1500 | |
| Tyrosine | — | — | 371 | | | | 371 | |
| Povidone | 26 | 26 | 26 | 26 | 26 | 26 | 26 | 26 |
| Stearic Acid | 10 | 10 | 10 | 10 | 10 | 10 | 10 | 10 |
| Other | | | | Glycine | Glycine | — | — | |
| | | | | 467 | 467 | | | |
| | | | | | Sodium glucuronate | | | |
| | | | | | 145 | | | |
| TOTAL | 991 | 2286 | 2657 | 1253 | 1398 | 1120 | 2862 | 786 |
| Absorption Onset | 1-2 | 0.3-0.5 | 0.4-0.6 | 0.3-0.5 | 0.3-0.5 | 1-2 | 1-2 | 0.3-0.5 |
| (Phase I) (hours) | | | | | | | | |
| Peak Dose (hours) | 5-12 | 1-2 | 1-2 | 1-2 | 1-2 | 5-12 | 17-33 | 1-2 |
| Steady State Excretion | 10-17 | 15-29 | 17-33 | 6-14 | 5-14 | 9-16 | | 9-17 |
| Half-life (hours) | | | | | | | | |

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A therapeutic composition comprising:
a salicylate administered in concert with at least one salicylate excretion product selected from the group consisting of: a glycine salicylate conjugate and a glucuronide salicylate conjugate.

2. The composition of claim 1 wherein said salicylate and said at least one salicylate excretion product are co-mixed.

3. The composition of claim 1 wherein said composition is in the form of a solid oral delivery dose.

4. The composition of claim 3 further comprising a swelling agent present in a quantity sufficient to increase the time of peak blood serum level to greater than four hours.

5. The composition of claim 4 wherein said swelling agent is a hydroxy C₁₋₈ alkyl C₁₋₈ alkyl cellulose.

6. The composition of claim 1 wherein said salicylate is selected from the group consisting of: alkali metal ion salicylates; alkali earth ion salicylates; transition metal salicylates; aluminum salicylate; quaternary alkyl ammoniums salicylates; choline salicylate; amino acid salicylates; oligopeptides salicylates; heterocyclic salicylates; choline magnesium salicylate; and choline magnesium trisalicylate; and combinations thereof.

7. The composition of claim 1 wherein said salicylate is a choline magnesium salicylate.

8. The composition of claim 1 further comprising an enzymatic substrate competitor present in a quantity to extend the excretion half-life of said salicylate, said enzymatic substrate competitor selected from the group consisting of: a benzoate derivative and an active phenolic unit.

9. The composition of claim 8 wherein said enzymatic substrate competitor is enterically coated.

10. A therapeutic composition comprising: a salicylate administered in concert with an enzymatic substrate competitor present in sufficient quantity to extend the excretion half-life of said salicylate relative to the absence of said competitor.

11. The composition of claim 10 wherein said competitor is a benzoate or derivative thereof.

12. The composition of claim 11 wherein said benzoate derivative is present in a molar stoichiometric ratio to said salicylate of 0.3-10:1.

13. The composition of claim 10 wherein said salicylate is one or more salicylates selected from the group consisting of: alkali metal ion salicylates; alkali earth ion salicylates; transition metal salicylates; aluminum salicylate; quaternary alkyl ammoniums salicylates; choline salicylate; amino acid salicylates; oligopeptides salicylates; heterocyclic salicylates; choline magnesium salicylate; and choline magnesium trisalicylate; and combinations thereof.

14. The composition of claim 12 wherein said salicylate has an excretion half-life of greater than 18 hours.

15. The composition of claim 14 wherein said salicylate is a choline magnesium salicylate.

16. The composition of claim 10 in a solid oral delivery dose further comprising a swelling agent.

17. A therapeutic composition comprising:
a choline magnesium salicylate; and
a hydrophilic matrix polymer swelling in gastric fluid to form a gel layer through which said choline magnesium salicylate diffuses.

18. The composition of claim 17 wherein said hydrophilic matrix polymer is a hydroxy C₁₋₈ alkyl C₁₋₈ alkyl cellulose.

19. The composition of claim 17 further comprising a tablet-pressing agent.

20. The composition of claim 17 further comprising a salicylate excretion product selected from the group consisting of: a glycine salicylate conjugate and a glucuronide salicylate conjugate.

21. The composition of claim 17 further comprising an enzymatic substrate competitor selected from the group consisting of: benzoate, a benzoate derivative and an active phenolic unit.

22. A process for treating a human or nonhuman subject comprising administering to the subject a therapeutically effective amount of a salicylate; and a salicylate pharmacokinetics modifier selected from the group consisting of: glycine salicylate conjugate, glucuronide salicylate conjugate, a benzoate, an active phenolic unit and a benzoate derivative; and a pharmaceutically acceptable carrier.

23. The process of claim 22 wherein administration is by a route selected from the group consisting of: intravenously, intrathecally, intraventricularly, intramuscularly, nasally, topically, and anally.

24. The process of claim 22 further comprising incorporating said composition into a transdermal patch.

25. The process of claim 22 wherein the subject is a human adult and between 300 and 900 milligrams per day are delivered.
